SEQUENCE LISTING (78 sequences)

SEQ ID NO: 1 - protein, 8 aa, artificial
  Consensus CDR -H1
  MISC_FEATURE (3)..(3): Thr may be replaced by Ser
  MISC_FEATURE (8)..(8): Tyr may be replaced by Phe
GYTFTSYY

SEQ ID NO: 2 - protein, 8 aa, artificial
  Consensus CDR-H2
IWPGDGST

SEQ ID NO: 3 - protein, 13 aa, artificial
  Consensus CDR-H3
ASPMITPNYAMDY

SEQ ID NO: 4 - protein, 6 aa, artificial
  Consensus CDR-L1
  MISC_FEATURE (4)..(4): Ser may be replaced by Asn
QDISKY

SEQ ID NO: 5 - protein, 3 aa, artificial
  Consensus CDR-L2
YTS

SEQ ID NO: 6 - protein, 9 aa, artificial
  Consensus CDR-L3
  MISC_FEATURE (5)..(5): Thr may be replaced by Ala
QQGSTLPYT

SEQ ID NO: 7 - protein, 8 aa, artificial
  CDR-H1
GYTFTSYY

SEQ ID NO: 8 - protein, 8 aa, artificial
  CDR-H1
GYSFTSYF

SEQ ID NO: 9 - protein, 6 aa, artificial
  CDR-L1
QDISKY

SEQ ID NO: 10 - protein, 6 aa, artificial
  CDR-L1
QDINKY

SEQ ID NO: 11 - protein, 9 aa, artificial
  CDR-L3
QQGSTLPYT

SEQ ID NO: 12 - protein, 9 aa, artificial
  CDR-L3
QQGSALPYT

SEQ ID NO: 13 - protein, 120 aa, artificial
  c208F2, heavy chain, VH
QVQLQQSGPELVKPGASVKMSCKASGYTFTSYYIYWVKQRPGQGLEWLGWIWPGDGSTKYNEKFKDKTTLTADKSSNTAYMFLSSLTSEDSAVYFCASPMITPNYAMDYWGQGASVTVSS

SEQ ID NO: 14 - protein, 120 aa, artificial
  c212A11, heavy chain, VH
QVQLQQSGPELVKPGASVKMSCKASGYTFTSYYIHWVKQRPGQGLEWIGWIWPGDGSTKYNEKFKGKTTLTADKSSSTAYMFLSSLTSEDSAVYFCASPMITPNYAMDYWGQGASVTVSS

SEQ ID NO: 15 - protein, 120 aa, artificial
  c214F8, heavy chain, VH
QVQLQQSGSELVKPGASVKLSCKASGYTFTSYYIHWVKQRPGQGLEWIGWIWPGDGSTKYNERFKGKTTLTADKSSSTAYMFLSSLTSEDSAVYFCASPMITPNYAMDYWGQGASVTVSS

SEQ ID NO: 16 - protein, 120 aa, artificial
  c219D6, heavy chain, VH
QVQLQQSGPELVKPGDSVKMSCKASGYSFTSYFIHWVKQRPGQGLEWIGWIWPGDGSTKYNEKFKGKTTLTTDKSSSTAYMFLNSLTSEDSAVYFCASPMITPNYAMDYWGQGASVTVSS

SEQ ID NO: 17 - protein, 120 aa, artificial
  c213B10, heavy chain, VH
QVQLQQSGSELVKPGASVKMSCKASGYTFTSYYIHWVKQRPGQGLEWIGWIWPGDGSTKYNERFKGKTTLTADKSSSTAYMFLSSLTSEDSAVYFCASPMITPNYAMDYWGQGASVTVSS

SEQ ID NO: 18 - protein, 107 aa, artificial
  c208F2, light chain, VL
DIQMTQTTSSLSASLGDRVTISCRASQDISKYLNWYQQQPDGTIKLLIYYTSRLHSGVPSRFSGRGSGTDYSLTISNVEQEDIATYFCQQGSTLPYTFGGGTKLEIK

SEQ ID NO: 19 - protein, 107 aa, artificial
  c212A11, light chain, VL
DIQMTQTTSSLSASLGDRVTISCRASQDINKYLNWYQQQPDGTVKLLIYYTSRLHSGVPSRFSGRGSGTDYSLTISNLEQEDIATYFCQQGSTLPYTFGGGTKLEIK

SEQ ID NO: 20 - protein, 107 aa, artificial
  c214F8, light chain, VL
DIQMTQTTSSLSASLGDRVTFSCRASQDISKYLNWYQQQPDGTIKLLIYYTSRLHSGVPSRFSGRGSGTDYSLTITNLEQEDIATYFCQQGSALPYTFGGGTKLEIK

SEQ ID NO: 21 - protein, 107 aa, artificial
  c219D6, light chain, VL
DIQMTQTTSSLSASLGDRVTISCRASQDISKYLNWYQQQPDGTVKLLIYYTSRLHSGVPSRFSGRGSGTDYSLTISNLEQEDIATYFCQQGSTLPYTFGGGTKLEIK

SEQ ID NO: 22 - protein, 107 aa, artificial
  c213B10, light chain, VL
DIQMTQTTSSLSASLGDRVTISCRASQDISKYLNWYQQQPDGTIKLLIYYTSRLHSGVPSRFSGRGSGTDYSLTITNLEQEDIATYFCQQGSALPYTFGGGTKLEIK

SEQ ID NO: 23 - protein, 449 aa, artificial
  c208F2, heavy chain, full length
QVQLQQSGPELVKPGASVKMSCKASGYTFTSYYIYWVKQRPGQGLEWLGWIWPGDGSTKYNEKFKDKTTLTADKSSNTAYMFLSSLTSEDSAVYFCASPMITPNYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 449 aa, artificial
  c212A11, heavy chain, full length
QVQLQQSGPELVKPGASVKMSCKASGYTFTSYYIHWVKQRPGQGLEWIGWIWPGDGSTKYNEKFKGKTTLTADKSSSTAYMFLSSLTSEDSAVYFCASPMITPNYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 449 aa, artificial
  c214F8, heavy chain, full length
QVQLQQSGSELVKPGASVKLSCKASGYTFTSYYIHWVKQRPGQGLEWIGWIWPGDGSTKYNERFKGKTTLTADKSSSTAYMFLSSLTSEDSAVYFCASPMITPNYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 449 aa, artificial
  c219D6, heavy chain, full length
QVQLQQSGPELVKPGDSVKMSCKASGYSFTSYFIHWVKQRPGQGLEWIGWIWPGDGSTKYNEKFKGKTTLTTDKSSSTAYMFLNSLTSEDSAVYFCASPMITPNYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 449 aa, artificial
  c213B10, heavy chain, full length
QVQLQQSGSELVKPGASVKMSCKASGYTFTSYYIHWVKQRPGQGLEWIGWIWPGDGSTKYNERFKGKTTLTADKSSSTAYMFLSSLTSEDSAVYFCASPMITPNYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 214 aa, artificial
  c208F2, light chain, full length
DIQMTQTTSSLSASLGDRVTISCRASQDISKYLNWYQQQPDGTIKLLIYYTSRLHSGVPSRFSGRGSGTDYSLTISNVEQEDIATYFCQQGSTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 214 aa, artificial
  c212A11, light chain, full length
DIQMTQTTSSLSASLGDRVTISCRASQDINKYLNWYQQQPDGTVKLLIYYTSRLHSGVPSRFSGRGSGTDYSLTISNLEQEDIATYFCQQGSTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 214 aa, artificial
  c214F8, light chain, full length
DIQMTQTTSSLSASLGDRVTFSCRASQDISKYLNWYQQQPDGTIKLLIYYTSRLHSGVPSRFSGRGSGTDYSLTITNLEQEDIATYFCQQGSALPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 214 aa, artificial
  c219D6, light chain, full length
DIQMTQTTSSLSASLGDRVTISCRASQDISKYLNWYQQQPDGTVKLLIYYTSRLHSGVPSRFSGRGSGTDYSLTISNLEQEDIATYFCQQGSTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 214 aa, artificial
  c213B10, light chain, full length
DIQMTQTTSSLSASLGDRVTISCRASQDISKYLNWYQQQPDGTIKLLIYYTSRLHSGVPSRFSGRGSGTDYSLTITNLEQEDIATYFCQQGSALPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 120 aa, artificial
  hz208F2 (var.1) heavy chain, VH
QVQLVQSGAEVKKPGASVKVSCKASGYTFTSYYMHWVRQAPGQGLEWMGIIWPGDGSTSYAQKFQGRVTMTRDTSTSTVYMELSSLRSEDTAVYYCASPMITPNYAMDYWGQGTLVTVSS

SEQ ID NO: 34 - protein, 120 aa, artificial
  hz208F2 (var. 3), VH
QVQLVQSGAEVKKPGASVKMSCKASGYTFTSYYIYWVKQAPGQGLEWLGWIWPGDGSTKYNEKFQGRVTLTADKSSNTAYMFLSSLRSEDTAVYFCASPMITPNYAMDYWGQGTLVTVSS

SEQ ID NO: 35 - protein, 107 aa, artificial
  hz208F2 (var. 1), VL
DIQMTQSPSSLSASVGDRVTITCRASQDISKYLNWYQQKPGKAPKLLIYYTSSLQSGVPSRFSGSGSGTDFTLTISSLQPEDFATYYCQQGSTLPYTFGGGTKVEIK

SEQ ID NO: 36 - protein, 107 aa, artificial
  hz208F2 (var.3), VL
DIQMTQSPSSLSASVGDRVTISCRASQDISKYLNWYQQKPGKAPKLLIYYTSRLHSGVPSRFSGRGSGTDYSLTISNLQPEDFATYFCQQGSTLPYTFGGGTKVEIK

SEQ ID NO: 37 - protein, 449 aa, artificial
  hz208F2 (var. 1), heavy chain, full length
QVQLVQSGAEVKKPGASVKVSCKASGYTFTSYYMHWVRQAPGQGLEWMGIIWPGDGSTSYAQKFQGRVTMTRDTSTSTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 449 aa, artificial
  hz208F2 (var.3), heavy chain full length
QVQLVQSGAEVKKPGASVKMSCKASGYTFTSYYIYWVKQAPGQGLEWLGWIWPGDGSTKYNEKFQGRVTLTADKSSNTAYMFLSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 214 aa, artificial
  hz208F2 (var. 1), light chain, full length
DIQMTQSPSSLSASVGDRVTITCRASQDISKYLNWYQQKPGKAPKLLIYYTSSLQSGVPSRFSGSGSGTDFTLTISSLQPEDFATYYCQQGSTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 214 aa, artificial
  hz208F2 (var.3), light chain, full length
DIQMTQSPSSLSASVGDRVTISCRASQDISKYLNWYQQKPGKAPKLLIYYTSRLHSGVPSRFSGRGSGTDYSLTISNLQPEDFATYFCQQGSTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 120 aa, artificial; identical to SEQ ID NO: 34
  hz208F2 (var.2) heavy chain, VH
  MISC_FEATURE (20)..(20): Met may be replaced by Val
  MISC_FEATURE (34)..(34): Ile may be replaced by Met
  MISC_FEATURE (35)..(35): Tyr may be replaced by His
  MISC_FEATURE (38)..(38): Lys may be replaced by Arg
  MISC_FEATURE (48)..(48): Leu may be replaced by Met
  MISC_FEATURE (50)..(50): Trp may be replaced by Ile
  MISC_FEATURE (59)..(59): Lys may be replaced by Ser
  MISC_FEATURE (61)..(61): Asn may be replaced by Ala
  MISC_FEATURE (62)..(62): Glu may be replaced by Gln
  MISC_FEATURE (70)..(70): Leu may be replaced by Met
  MISC_FEATURE (72)..(72): Ala may be replaced by Arg
  MISC_FEATURE (74)..(74): Lys may be replaced by Thr
  MISC_FEATURE (76)..(76): Ser may be replaced by Thr
  MISC_FEATURE (77)..(77): Asn may be replaced by Ser
  MISC_FEATURE (79)..(79): Ala may be replaced by Val
  MISC_FEATURE (82)..(82): Phe may be replaced by Glu
  MISC_FEATURE (95)..(95): Phe may be replaced by Tyr

SEQ ID NO: 42 - protein, 107 aa, artificial; identical to SEQ ID NO: 36
  hz208F2 (var. 2), light chain, VL
  MISC_FEATURE (22)..(22): Ser may be replaced by Thr
  MISC_FEATURE (53)..(53): Arg may be replaced by Ser
  MISC_FEATURE (55)..(55): His may be replaced by Gln
  MISC_FEATURE (65)..(65): Arg may be replaced by Ser
  MISC_FEATURE (71)..(71): Tyr may be replaced by Phe
  MISC_FEATURE (72)..(72): Ser may be replaced by Thr
  MISC_FEATURE (77)..(77): Asn may be replaced by Ser
  MISC_FEATURE (87)..(87): Phe may be replaced by Tyr

SEQ ID NO: 43 - protein, 329 aa, artificial
  Constant domain (VH) IgG1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 326 aa, artificial
  Constant domain (VH) IgG4 (S228P)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 45 - protein, 107 aa, artificial [sequence omitted]

SEQ ID NO: 46 - protein, 98 aa, artificial
  Human germline IGHV1-46*01
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 47 - protein, 95 aa, artificial
  Human germline IGKV1-39*01
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 48 - protein, 15 aa, artificial
  Human germline IGHJ4*01
YFDYWGQGTLVTVSS

SEQ ID NO: 49 - protein, 12 aa, artificial
  Human germline IGKJ4*01
LTFGGGTKVEIK

SEQ ID NO: 50 - protein, 1367 aa, artificial
  IGF-1R (human)
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 51 - protein, 932 aa, artificial
  IGF-1R ECD (human)
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYEN

SEQ ID NO: 52 - protein, 512 aa, artificial
  IGF-1R ECD Nterminal (human)
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRY

SEQ ID NO: 53 - protein, 449 aa, artificial
  hz208F2 heavy chain H076 full length
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 120 aa, artificial
  hz208F2 heavy chain H077, VH
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSS

SEQ ID NO: 55 - protein, 449 aa, artificial
  hz208F2 heavy chain H077 full length
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 120 aa, artificial
  hz208F2 heavy chain H037, VH
QVQLVQSGAEVKKPGASVKVSCKASGYTFTSYYMHWVRQAPGQGLEWMGWIWPGDGSTKYAQKFQGRVTMTRDKSSSTVYMELSSLRSEDTAVYFCASPMITPNYAMDYWGQGTLVTVSS

SEQ ID NO: 57 - protein, 107 aa, artificial
  hz208F2 light chain L018, VL
DIQMTQSPSSLSASVGDRVTITCRASQDISKYLNWYQQKPGKAPKLLIYYTSRLQSGVPSRFSGRGSGTDYSLTISSLQPEDFATYFCQQGSTLPYTFGGGTKVEIK

SEQ ID NO: 58 - protein, 449 aa, artificial
  hz208F2 heavy chain H037 full length
QVQLVQSGAEVKKPGASVKVSCKASGYTFTSYYMHWVRQAPGQGLEWMGWIWPGDGSTKYAQKFQGRVTMTRDKSSSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 214 aa, artificial
  hz208F2 light chain L018 full length
DIQMTQSPSSLSASVGDRVTITCRASQDISKYLNWYQQKPGKAPKLLIYYTSRLQSGVPSRFSGRGSGTDYSLTISSLQPEDFATYFCQQGSTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 107 aa, artificial
  hz208F2 light chain L021, VL
DIQMTQSPSSLSASVGDRVTITCRASQDISKYLNWYQQKPGKAPKLLIYYTSRLHSGVPSRFSGRGSGTDFSLTISSLQPEDFATYFCQQGSTLPYTFGGGTKVEIK

SEQ ID NO: 61 - protein, 214 aa, artificial
  hz208F2 light chain L021 full length
DIQMTQSPSSLSASVGDRVTITCRASQDISKYLNWYQQKPGKAPKLLIYYTSRLHSGVPSRFSGRGSGTDFSLTISSLQPEDFATYFCQQGSTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 120 aa, artificial
  hz208F2 heavy chain H047, VH
QVQLVQSGAEVKKPGASVKVSCKASGYTFTSYYIHWVRQAPGQGLEWMGWIWPGDGSTKYAQKFQGRVTMTRDTSSNTVYMELSSLRSEDTAVYYCASPMITPNYAMDYWGQGTLVTVSS

SEQ ID NO: 63 - protein, 449 aa, artificial
  hz208F2 heavy chain H047 full length
QVQLVQSGAEVKKPGASVKVSCKASGYTFTSYYIHWVRQAPGQGLEWMGWIWPGDGSTKYAQKFQGRVTMTRDTSSNTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 120 aa, artificial
  hz208F2 heavy chain H049, VH
QVQLVQSGAEVKKPGASVKVSCKASGYTFTSYYMHWVRQAPGQGLEWMGWIWPGDGSTKYAQKFQGRVTMTRDKSSSTVYMELSSLRSEDTAVYYCASPMITPNYAMDYWGQGTLVTVSS

SEQ ID NO: 65 - protein, 449 aa, artificial
  hz208F2 heavy chain H049 full length
QVQLVQSGAEVKKPGASVKVSCKASGYTFTSYYMHWVRQAPGQGLEWMGWIWPGDGSTKYAQKFQGRVTMTRDKSSSTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 120 aa, artificial
  hz208F2 heavy chain H051, VH
QVQLVQSGAEVKKPGASVKVSCKASGYTFTSYYMHWVRQAPGQGLEWMGWIWPGDGSTKYAQKFQGRVTMTRDTSSNTVYMELSSLRSEDTAVYFCASPMITPNYAMDYWGQGTLVTVSS

SEQ ID NO: 67 - protein, 449 aa, artificial
  hz208F2 heavy chain H051 full length
QVQLVQSGAEVKKPGASVKVSCKASGYTFTSYYMHWVRQAPGQGLEWMGWIWPGDGSTKYAQKFQGRVTMTRDTSSNTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 120 aa, artificial
  hz208F2 heavy chain H052, VH
QVQLVQSGAEVKKPGASVKVSCKASGYTFTSYYMHWVRQAPGQGLEWMGWIWPGDGSTKYAQKFQGRVTMTRDTSTSTVYMELSSLRSEDTAVYFCASPMITPNYAMDYWGQGTLVTVSS

SEQ ID NO: 69 - protein, 449 aa, artificial
  hz208F2 heavy chain H052 full length
QVQLVQSGAEVKKPGASVKVSCKASGYTFTSYYMHWVRQAPGQGLEWMGWIWPGDGSTKYAQKFQGRVTMTRDTSTSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 120 aa, artificial
  hz208F2 heavy chain H057, VH
QVQLVQSGAEVKKPGASVKVSCKASGYTFTSYYIHWVRQAPGQGLEWMGWIWPGDGSTKYAQKFQGRVTMTRDKSTNTVYMELSSLRSEDTAVYYCASPMITPNYAMDYWGQGTLVTVSS

SEQ ID NO: 71 - protein, 449 aa, artificial
  hz208F2 heavy chain H057 full length
QVQLVQSGAEVKKPGASVKVSCKASGYTFTSYYIHWVRQAPGQGLEWMGWIWPGDGSTKYAQKFQGRVTMTRDKSTNTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 120 aa, artificial
  hz208F2 heavy chain H068, VH
QVQLVQSGAEVKKPGASVKVSCKASGYTFTSYYMHWVRQAPGQGLEWMGWIWPGDGSTKYAQKFQGRVTMTRDTSTNTVYMELSSLRSEDTAVYFCASPMITPNYAMDYWGQGTLVTVSS

SEQ ID NO: 73 - protein, 449 aa, artificial
  hz208F2 heavy chain H068 full length
QVQLVQSGAEVKKPGASVKVSCKASGYTFTSYYMHWVRQAPGQGLEWMGWIWPGDGSTKYAQKFQGRVTMTRDTSTNTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 120 aa, artificial
  hz208F2 heavy chain H070, VH
QVQLVQSGAEVKKPGASVKVSCKASGYTFTSYYMHWVRQAPGQGLEWMGWIWPGDGSTKYAQKFQGRVTMTRDTSTSTVYMELSSLRSEDTAVYYCASPMITPNYAMDYWGQGTLVTVSS

SEQ ID NO: 75 - protein, 449 aa, artificial
  hz208F2 heavy chain H070 full length
QVQLVQSGAEVKKPGASVKVSCKASGYTFTSYYMHWVRQAPGQGLEWMGWIWPGDGSTKYAQKFQGRVTMTRDTSTSTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 120 aa, artificial
  hz208F2 heavy chain H071, VH
QVQLVQSGAEVKKPGASVKVSCKASGYTFTSYYMHWVRQAPGQGLEWMGWIWPGDGSTKYAQKFQGRVTMTRDTSTNTVYMELSSLRSEDTAVYYCASPMITPNYAMDYWGQGTLVTVSS

SEQ ID NO: 77 - protein, 449 aa, artificial
  hz208F2 heavy chain H071 full length
QVQLVQSGAEVKKPGASVKVSCKASGYTFTSYYMHWVRQAPGQGLEWMGWIWPGDGSTKYAQKFQGRVTMTRDTSTNTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 120 aa, artificial
  hz208F2 heavy chain H076, VH
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYYCASPMITPNYAMDYWGQGTLVTVSS

(19) United States
(12) Patent Application Publication
(10) Pub. No.: US 2022/0218838 A1
(43) Pub. Date: Jul. 14, 2022

(54) ADC FOR A TREATMENT CONCOMITANT WITH OR SUBSEQUENT TO DOCETAXEL

(71) Applicant: PIERRE FABRE MEDICAMENT, Boulogne-Billancourt (FR)

(72) Inventors: Alexandra JOUHANNEAUD, Montpellier (FR); Liliane GOETSCH, Ayze (FR)

(73) Assignee: PIERRE FABRE MEDICAMENT, Boulogne-Billancourt (FR)

(21) Appl. No.: 17/609,129

(22) PCT Filed: May 6, 2020
(86) PCT No.: PCT/EP2020/062520

§ 371 Date: Nov. 5, 2021

(30) Foreign Application Priority Data
May 6, 2019 (EP) 19305578.7

(51) Int. Cl. A61K 47/68; A61P 35/00; A61K 47/54; A61K 31/704; A61K 31/675; A61K 31/7068; A61K 33/243; A61K 31/337; A61K 31/506; A61K 31/282; A61K 31/513; A61K 31/65; A61K 31/4184

(52) U.S. Cl. CPC A61K 47/6849; A61K 47/6817; A61P 35/00; A61K 47/545; A61K 31/704; A61K 31/675; A61K 31/4184; A61K 33/243; A61K 31/337; A61K 31/506; A61K 31/282; A61K 31/513; A61K 31/65; A61K 31/7068

(57) ABSTRACT

The present invention relates to an ADC having the following formula (I) in which Ab is an anti-IGF1R antibody characterized in that it comprises light chain CDRs of sequences SEQ ID Nos. 9, 5 and 11, respectively, and heavy chain CDRs of sequences SEQ ID Nos. 7, 2 and 3, respectively, and n is between 1 and 12, for use in the treatment of proliferative diseases, such as cancer, characterized in that the ADC is administered concomitantly with or subsequent to docetaxel.

The present invention relates to the use of an anti-IGF1R ADC (ADC-A) for the treatment of proliferative diseases, such as cancer, characterised in that ADC-A is administered concomitantly with or subsequently to docetaxel.

Hyperproliferative diseases, such as cancer, are a real public health challenge. Obviously, there is a need to find effective first-line anti-tumour drugs. But there is also a need to provide long-term solutions in the event resistance to these first line treatments appears, which is a common phenomenon.

Docetaxel is a diterpene used as a cancer medicament that acts on cancer cell microtubules and results in blocking mitosis. The effectiveness of docetaxel has been proven in a fairly large number of tumours, especially in breast cancer, lung cancer and prostate cancer. However, a large number of patients develop resistance to docetaxel over time, leading to treatment failure.

The insulin-like growth-factor 1 receptor, called IGF-1R (or IGF1R) is a receptor with tyrosine kinase activity having a homology of 70% with the insulin receptor IR. IGF-IR is a glycoprotein with a molecular weight of approximately 350,000. It is a heterotetramer receptor for which each moiety—linked by disulfide bridges—is comprised of an extracellular a subunit and a transmembrane 3 subunit. IGF-IR binds IGF1 and IGF2 with a very high affinity (Kd #1 nM) and is able to bind insulin with an affinity 100 to 1000 times less.

Cytoplasmic tyrosine kinase proteins are activated by binding of the ligand to the extracellular domain of IGF-1R. The activation of these kinases leads in turn to the inactivation of various intracellular substrates, including IRS-1, IRS-2, She and Grb 10. Via the activation of numerous downstream effectors, IRS proteins are involved in numerous pathways inducing cell growth and differentiation. Moreover, it appears that the pathway mainly involved in protection against apoptosis is the phosphatidyl-inositol 3-kinase (Pi3-kinase) pathway.

The role of the IGF system in carcinogenesis has been the subject of intensive research over the past ten years. Indeed, it is well established that activation of the IGF-IR leads, in a large variety of cells, to an abnormal growth of independent cells and to tumour formation. The IGF-IR is thus expressed in a large variety of tumours and tumour lines.

In order to treat this type of cancer, treatments targeting the IGF1R have been developed. It has thus been established that the use of an ADC (antibody-drug conjugate) targeting the IGF-1R makes it possible to reduce and even abolish tumour growth. Such ADCs have notably been described in WO2015162291, in particular the ADC described below, called ADC-A.

There is a real need for effective treatment for hyperproliferative diseases. It is therefore necessary to identify combinations of therapies to make it possible to escape the onset of the phenomenon of tumour resistance.

Surprisingly, the present invention showed that the use of an anti-IGF1R ADC in combination with docetaxel leads to a synergistic effect in tumour treatment. Also surprisingly, it was shown that the use of an anti-IGF1R ADC made it possible to treat tumours that became resistant after docetaxel treatment.

Figure Legend:

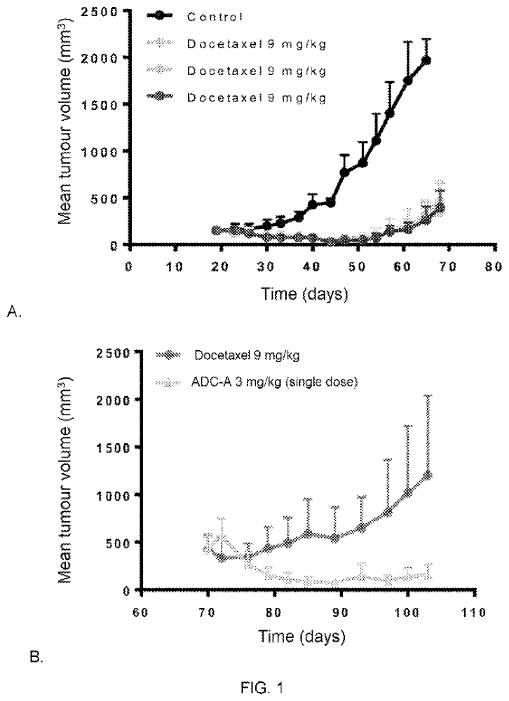

FIG. 1: Progression of mean tumour volume in mm³ as a function of time in days in mice bearing an MCF-7 tumour of approximately 150 mm³ after A. Treatment with docetaxel at 9 mg/kg once every two weeks for 5 cycles (the three merged curves are three series of 5 mice treated identically and at the same time) and B. Treatment of mice who became resistant to docetaxel on day 70 by: group 1, docetaxel 9 mg/kg every two weeks (circles) and; group 2, an injection of ADC-A at 3 mg/kg (triangles).

The present invention therefore has for an object an anti-IGF1R ADC (ADC-A) for use in proliferative diseases, such as cancer, characterised in that ADC-A is administered concomitantly with or subsequently to docetaxel.

The present invention also has for an object the use of an anti-IGF1R ADC (ADC-A) for the treatment of proliferative diseases, such as cancer, characterised in that ADC-A is administered concomitantly with or subsequently to docetaxel.

The present invention also has for an object the use of an anti-IGF1R ADC (ADC-A) for the preparation of a medicament intended for the treatment of proliferative diseases, such as cancer, characterised in that ADC-A is administered concomitantly with or subsequently to docetaxel.

The present invention also has for an object a method for treating a proliferative disease, such as cancer, comprising the administration to a patient in need thereof of an effective quantity of an anti-IGF1R ADC (ADC-A) and docetaxel, characterised in that ADC-A is administered concomitantly with or subsequently to docetaxel.

Docetaxel

According to the invention, docetaxel means the compound of the following formula:

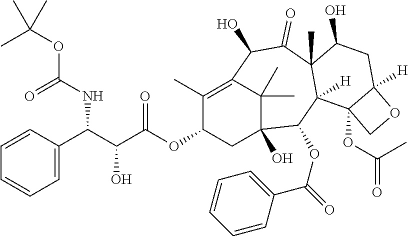

as well as its pharmaceutically-acceptable salts and solvates, especially its trihydrate.

Docetaxel is especially sold under the brand name Taxotere®.

In the present invention, “pharmaceutically acceptable” means that which is useful in the preparation of a pharmaceutical compound, which is generally safe, nontoxic and not biologically or otherwise undesirable and which is acceptable for both veterinary and human pharmaceutical use.

“Pharmaceutically-acceptable salt or solvate” of a compound means a salt or solvate that is pharmaceutically acceptable, such as defined here, and which has the desired pharmacological activity of the parent compound.

Pharmaceutically-acceptable salts notably include:

(1) pharmaceutically-acceptable acid addition salts formed with pharmaceutically-acceptable inorganic acids such as hydrochloric acid, hydrobromic acid, sulfuric acid, nitric acid, phosphoric acid and the like; or formed with pharmaceutically-acceptable organic acids such as formic acid, acetic acid, benzenesulfonic acid, benzoic acid, camphorsulfonic acid, citric acid, ethanesulfonic acid, fumaric acid, glucoheptanoic acid, gluconic acid, glutamic acid, glycolic acid, hydroxy naphthoic acid, 2-hydroxy ethanesulfonic acid, lactic acid, maleic acid, malic acid, mandelic acid, methanesulfonic acid, muconic acid, 2-naphthalene sulfonic acid, propionic acid, salicylic acid, succinic acid, dibenzoyl-L-tartaric acid, tartaric acid, p-toluenesulfonic acid, trimethylacetic acid, trifluoroacetic acid and the like, and

(2) pharmaceutically-acceptable base addition salts formed when an acidic proton present in the parent compound is either replaced by a metal ion, for example an alkali metal iron, an alkaline-earth metal ion or an aluminium; or is coordinated with a pharmaceutically-acceptable organic base such as diethanolamine, ethanolamine, N-methylglucamine, triethanolamine, tromethamine and the like; or with a pharmaceutically-acceptable inorganic base such as aluminium hydroxide, calcium hydroxide, potassium hydroxide, sodium carbonate, sodium hydroxide and the like.

Acceptable solvates for the pharmaceutical use of compounds according to the present invention include standard solvates such as those formed during the last step of the preparation method for the compounds according to the invention with the reaction solvent(s). Solvates formed with water (commonly called hydrates) or with ethanol can be mentioned as examples. For example, it can be a trihydrate.

ADC-A

ADC-A is an anti-IGF1R ADC of the following formula

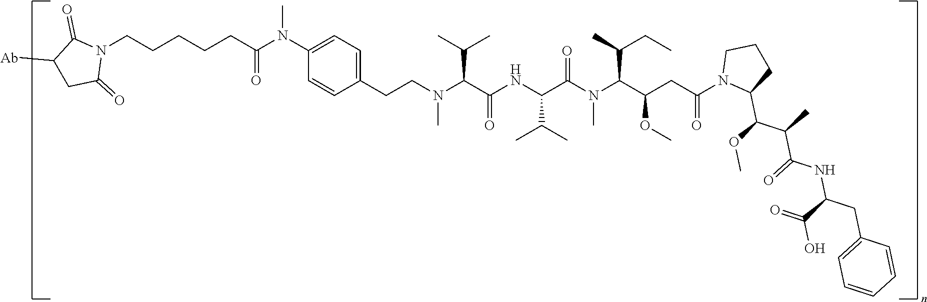

in which Ab is an anti-IGF1R antibody comprising three heavy chain complementarity determining regions (CDRs) of sequences SEQ ID No. 1, 2 and 3 and three light chain CDRs of sequences SEQ ID No. 4, 5 and 6, and n is comprised between 1 and 12, in particular, is equal to 2 or 4.

The terms “antibody”, “ab”, “Ab”, “mAb” or “immunoglobulin” are used interchangeably in the broadest sense and comprise monoclonal antibodies, modified or recombinant isolated antibodies (for example, complete or intact monoclonal antibodies), polyclonal antibodies, multivalent antibodies or multispecific antibodies (for example bispecific antibodies) as well as antibody fragments thereof, so that they have the desired biological activity, in particular the antigen binding fragments thereof. More particularly, it is a monoclonal antibodies or possibly the antigen binding fragments thereof.

The term “antigen binding fragment” of an antibody according to the invention is intended to indicate any peptide, polypeptide or protein conserving the aptitude of the antibody to bind to the target (generally called antigen).

As used in the present description, the expression “anti-IGF-1R antibody” designates an antibody able to bind IGF-1R, more particularly to an epitope located in IGF-1R, preferably the extracellular domain of IGF-1R, more preferably human IGF-1R (SEQ ID No. 50) and still more preferably the extracellular domain of human IGF-1R (SEQ ID No. 51), and even more preferably, the N-terminal part of the extracellular domain of human IGF-1R (SEQ ID No. 52), or any one of its natural variant sequences.

According to a particular embodiment of the invention, the antibody according to the invention comprises three heavy chain CDRs comprising or consisting of sequences SEQ ID No. 1, 2 and 3, or any sequence having at least 80%, preferably 85%, 90%, 95% and 98% identity with SEQ ID No. 1, 2 or 3; and three light chain CDRs comprising or consisting of sequences SEQ ID No. 4, 5 and 6, or any sequence having at least 80%, preferably 85%, 90%, 95% and 98% identity with SEQ ID No. 4, 5 or 6.

The percentages of identity referred to in the disclosure of the present invention are determined on the basis of a global alignment of sequences to be compared, i.e., on an alignment of sequences taken in their entirety over the entire length by using any algorithm well known to the person skilled in the art such as the algorithm of Needleman and Wunsch-1970. This comparison of sequences can be done by means of any software well known to the skilled person, for example Needle software using the “Gap open” parameter equal to 10.0, the “Gap extend” parameter equal to 0.5 and a “BLOSUM62” matrix. The Needle software is available, for example, on the ebi.ac.uk world wide website under the name “Align”.

When the antibody according to the invention has an amino acid sequence that is not 100% identical to the reference sequence but which has at least 80%, preferably 85%, 90%, 95%, and 98% identity with such a reference sequence, it can have insertions, deletions or substitutions with regard to the reference sequence. When it is a matter of substitutions, the substitution is preferably done with an “equivalent” amino acid, that is to say any amino acid whose structure is similar to the original amino acid and therefore is unlikely to change the biological activity of the antibody. Examples of such substitutions are presented in the following table:

| Original amino acid | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (G) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (VV) | Tyr |
| Tyr (Y) | Phe, Trp |
| Val (V) | Leu, Ala |

Table 1 below illustrates the CDR sequences for the preferred antibodies:

TABLE 1

| | Heavy chain | Light chain | SEQ ID No. |
|---|---|---|---|
| Consensus: | CDR-H1 | | 1 |
| | CDR-H2 | | 2 |
| | CDR-H3 | | 3 |
| | | CDR-L1 | 4 |
| | | CDR-L2 | 5 |
| | | CDR-L3 | 6 |
| 208F2 | CDR-H1 | | 7 |
| | CDR-H2 | | 2 |
| | CDR-H3 | | 3 |
| | | CDR-L1 | 9 |
| | | CDR-L2 | 5 |
| | | CDR-L3 | 11 |
| 212A11 | CDR-H1 | | 7 |
| | CDR-H2 | | 2 |
| | CDR-H3 | | 3 |
| | | CDR-L1 | 10 |
| | | CDR-L2 | 5 |
| | | CDR-L3 | 11 |
| 214F8 | CDR-H1 | | 7 |
| & | CDR-H2 | | 2 |
| 213B10 | CDR-H3 | | 3 |
| | | CDR-L1 | 9 |
| | | CDR-L2 | 5 |
| | | CDR-L3 | 12 |
| 219D6 | CDR-H1 | | 8 |
| | CDR-H2 | | 2 |
| | CDR-H3 | | 3 |
| | | CDR-L1 | 9 |
| | | CDR-L2 | 5 |
| | | CDR-L3 | 11 |

According to a specific aspect, the antibody is a mouse antibody characterised in that it also comprises light chain and heavy chain constant regions derived from an antibody of a heterologous species to mice, especially human.

According to a specific aspect of the invention, the antibody is a chimeric antibody (c) characterised in that it also comprises light chain and heavy chain constant regions derived from antibodies of a heterologous species with mice, especially human.

A chimeric antibody is an antibody containing a natural variable region (light chain and heavy chain) derived from an antibody of a given species in combination with light chain and heavy chain constant regions of an antibody of a species heterologous to said given species.

According to one embodiment of the invention, the antibody is chosen from among:

a) an antibody comprising three heavy chain CDRs of sequences SEQ ID No. 7, 2 and 3, and three light chain CDRs of sequences SEQ ID No. 9, 5 and 11;
b) an antibody comprising three heavy chain CDRs of sequences SEQ ID No. 7, 2 and 3 and three light chain CDRs of sequences SEQ ID No. 10, 5 and 11;
c) an antibody comprising three heavy chain CDRs of sequences SEQ ID No. 7, 2 and 3 and three light chain CDRs of sequences SEQ ID No. 9, 5 and 12; and
d) an antibody comprising three heavy chain CDRs of sequences SEQ ID No. 8, 2 and 3 and three light chain CDRs of sequences SEQ ID No. 9, 5 and 11.

In a preferred but non-limiting embodiment of the invention, the antibody is chosen from among:

a) an antibody comprising a heavy chain variable domain of sequence SEQ ID No. 13 or any sequence having at least 80% identity with SEQ ID No. 13 and three light chain CDRs of sequences SEQ ID No. 9, 5 and 11;
b) an antibody comprising a heavy chain variable domain of sequence SEQ ID No. 14 or any sequence having at least 80% identity with SEQ ID No. 14 and three light chain CDRs of sequences SEQ ID No. 10, 5 and 11;
c) an antibody comprising a heavy chain variable domain of sequence SEQ ID No. 15 or any sequence having at least 80% identity with SEQ ID No. 15 and three light chain CDRs of sequences SEQ ID No. 9, 5 and 12;
d) an antibody comprising a heavy chain variable domain of sequence SEQ ID No. 16 or any sequence having at least 80% identity with SEQ ID No. 16 and three light chain CDRs of sequences SEQ ID No. 9, 5 and 11; and
e) an antibody comprising a heavy chain variable domain of sequence SEQ ID No. 17 or any sequence having at least 80% identity with SEQ ID No. 17 and three light chain CDRs of sequences SEQ ID No. 9, 5 and 12.

“Any sequence having at least 80%, preferably 85%, 90%, 95% and 98% identity with SEQ ID No. 13 to 17” means, respectively, sequences having three heavy chain CDRs SEQ ID No. 1, 2 and 3 and, moreover, having at least 80%, preferably 85%, 90%, 95% and 98%, identity with complete sequence SEQ ID No. 13 to 17 beyond the sequences corresponding to the CDRs (i.e., SEQ ID No. 1, 2 and 3).

According to one embodiment of the invention, the antibody is chosen from among:

a) an antibody comprising a heavy chain variable domain of sequence SEQ ID No. 13, and three light chain CDRs of sequence SEQ ID No. 9, 5 and 11;
b) an antibody comprising a heavy chain variable domain of sequence SEQ ID No. 14 and three light chain CDRs of sequence SEQ ID No. 10, 5 and 11;
c) an antibody comprising a heavy chain variable domain of sequence SEQ ID No. 15 and three light chain CDRs of sequences SEQ ID No. 9, 5 and 12;
d) an antibody comprising a heavy chain variable domain of sequence SEQ ID No. 16, and three light chain CDRs of sequence SEQ ID No. 9, 5 and 11; and
e) an antibody comprising a heavy chain variable domain of sequence SEQ ID No. 17, and three light chain CDRs of sequence SEQ ID No. 9, 5 and 12.

In another preferred but non-limiting embodiment, the antibody is chosen from among:

a) an antibody comprising a light chain variable domain of sequence SEQ ID No. 18 or any sequence having at least 80% identity with SEQ ID No. 18 and three heavy chain CDRs of sequences SEQ ID No. 7, 2 and 3;
b) an antibody comprising a light chain variable domain of sequence SEQ ID No. 19 or any sequence having at least 80% identity with SEQ ID No. 19 and three heavy chain CDRs of sequences SEQ ID No. 7, 2 and 3;
c) an antibody comprising a light chain variable domain of sequence SEQ ID No. 20 or any sequence having at least 80% identity with SEQ ID No. 20 and three heavy chain CDRs of sequences SEQ ID No. 7, 2 and 3;
d) an antibody comprising a light chain variable domain of sequence SEQ ID No. 21 or any sequence having at least 80% identity with SEQ ID No. 21 and three heavy chain CDRs of sequences SEQ ID No. 8, 2 and 3; and
e) an antibody comprising a light chain variable domain of sequence SEQ ID No. 22 or any sequence having at least 80% identity with SEQ ID No. 22 and three heavy chain CDRs of sequences SEQ ID No. 7, 2 and 3.

“Any sequence having at least 80%, preferably 85%, 90%, 95% and 98% identity with SEQ ID No. 18 to 22” means, respectively, sequences having light chain CDRs SEQ ID No. 4, 5 and 6 and, moreover, having at least 80%, preferably 85%, 90%, 95% and 98%, identity with complete sequence SEQ ID No. 18 to 22 beyond the sequences corresponding to the CDRs (i.e., SEQ ID No. 4, 5 and 6).

According to one embodiment of the invention, the antibody is chosen from among:

a) an antibody comprising a light chain variable domain of sequence SEQ ID No. 18, and three heavy chain CDRs of sequence SEQ ID No. 7, 2 and 3;
b) an antibody comprising a light chain variable domain of sequence SEQ ID No. 19 and three heavy chain CDRs of sequence SEQ ID No. 7, 2 and 3;
c) an antibody comprising a light chain variable domain of sequence SEQ ID No. 20 and three heavy chain CDRs of sequence SEQ ID No. 7, 2 and 3;
d) an antibody comprising a light chain variable domain of sequence SEQ ID No. 21, and three heavy chain CDRs of sequence SEQ ID No. 8, 2 and 3; and
e) an antibody comprising a light chain variable domain of sequence SEQ ID No. 22 and three heavy chain CDRs of sequence SEQ ID No. 7, 2 and 3;

According to one embodiment of the invention, the antibody is an antibody chosen from among:

a) an antibody comprising a heavy chain variable domain of sequence SEQ ID No. 13 or any sequence having at least 80% identity with SEQ ID No. 13 and a light chain variable domain of sequence SEQ ID No. 18 or any sequence having at least 80% identity with SEQ ID No. 18;
b) an antibody comprising a heavy chain variable domain of sequence SEQ ID No. 14 or any sequence having at least 80% identity with SEQ ID No. 14 and a light chain variable domain of sequence SEQ ID No. 19, or any sequence having at least 80% identity with SEQ ID No. 19;
c) an antibody comprising a heavy chain variable domain of sequence SEQ ID No. 15 or any sequence having at least 80% identity with SEQ ID No. 15 and a light chain variable domain of sequence SEQ ID No. 20 or any sequence having at least 80% identity with SEQ ID No. 20;
d) an antibody comprising a heavy chain variable domain of sequence SEQ ID No. 16 or any sequence having at least 80% identity with SEQ ID No. 16 and a light chain variable domain of sequence SEQ ID No. 21 or any sequence having at least 80% identity with SEQ ID No. 21; and
e) an antibody comprising a heavy chain variable domain of sequence SEQ ID No. 17 or any sequence having at least 80% identity with SEQ ID No. 17 and a light chain variable domain of sequence SEQ ID No. 22, or any sequence having at least 80% identity with SEQ ID No. 22.

The chimeric antibodies described here can also be characterised by the constant domain and, more particularly, said chimeric antibodies can be selected or designed as such, without limitation, IgG1, IgG2, IgG3, IgM, IgA, IgD or IgE. More preferentially, in the context of the present invention, said chimeric antibodies are IgG1 or IgG4.

According to one embodiment of the invention, the antibody is a chimeric antibody comprising heavy chain variable domains (VH) and light chain variable domains (VL) such as described above in the IgG1 format. More preferentially, said chimeric antibody comprises a constant domain for the VH of sequence SEQ ID No. 43 and a kappa domain for the VL of sequence SEQ ID No. 45.

According to one embodiment of the invention, the antibody is a chimeric antibody comprising VH and VL variable domains such as described above in the IgG4 format. More preferentially, said chimeric antibody comprises a constant domain for the VH of sequence SEQ ID No. 44 and a kappa domain for the VL of sequence SEQ ID No. 45.

In another preferred but non-limiting embodiment, the antibody is chosen from among:

a) an antibody comprising or consisting of a heavy chain of sequence SEQ ID No. 23 or any sequence having at least 80% identity with SEQ ID No. 23 and a light chain of sequence SEQ ID No. 28 or any sequence having at least 80% identity with SEQ ID No. 28;
b) an antibody comprising or consisting of a heavy chain of sequence SEQ ID No. 24 or any sequence having at least 80% identity with SEQ ID No. 24 and a light chain of sequence SEQ ID No. 29 or any sequence having at least 80% identity with SEQ ID No. 29;
c) an antibody comprising or consisting of a heavy chain of sequence SEQ ID No. 25 or any sequence having at least 80% identity with SEQ ID No. 25 and a light chain of sequence SEQ ID No. 30 or any sequence having at least 80% identity with SEQ ID No. 30;
d) an antibody comprising or consisting of a heavy chain of sequence SEQ ID No. 26 or any sequence having at least 80% identity with SEQ ID No. 26 and a light chain of sequence SEQ ID No. 31 or any sequence having at least 80% identity with SEQ ID No. 31; and
e) an antibody comprising or consisting of a heavy chain of sequence SEQ ID No. 27 or any sequence having at least 80% identity with SEQ ID No. 27 and a light chain of sequence SEQ ID No. 32 or any sequence having at least 80% identity with SEQ ID No. 32.

For further clarity, Table 2 below illustrates the VH and VL sequences, respectively, for the preferred chimeric antibodies.

TABLE 2

| | Heavy chain | Light chain | SEQ ID No. |
|---|---|---|---|
| c208F2 | Variable domain (VH) | | 13 |
| | | Variable domain (VL) | 18 |
| | Complete length | | 23 |
| | | Complete length | 28 |
| c212A11 | Variable domain (VH) | | 14 |
| | | Variable domain (VL) | 19 |
| | Complete length | | 24 |
| | | Complete length | 29 |
| c214F8 | Variable domain (VH) | | 15 |
| | | Variable domain (VL) | 20 |
| | Complete length | | 25 |
| | | Complete length | 30 |
| c219D6 | Variable domain (VH) | | 16 |
| | | Variable domain (VL) | 21 |
| | Complete length | | 26 |
| | | Complete length | 31 |
| c213B10 | Variable domain (VH) | | 17 |
| | | Variable domain (VL) | 22 |
| | Complete length | | 27 |
| | | Complete length | 32 |

According to another specific aspect of the present invention, the antibody is a humanised antibody characterised in that the constant regions of the light chain and of the heavy chain derived from the human antibody are, respectively, the lambda or kappa region and the gamma-1, gamma-2 or gamma-4 regions.

In a preferred embodiment, the antibody comprises a heavy chain variable domain (VH) having:

i) CDR-H1, CDR-H2 and CDR-H3 of sequences SEQ ID No. 7, 2 and 3, respectively, and
ii) FR1, FR2 and FR3 derived from the human germ line IGHV1-46*01 (SEQ ID No. 46), and
iii) FR4 derived from the human germ line IGHJ4*01 (SEQ ID No. 48).

In a preferred embodiment, the antibody comprises a light chain variable domain (VL) having:

i) CDR-L1, CDR-L2 and CDR-L3 of sequences SEQ ID No. 9, 5 and 11, respectively, and
ii) FR1, FR2 and FR3 derived from the human germ line IGKV1-39*01 (SEQ ID No. 47), and
iii) FR4 derived from the human germ line IGKJ4*01 (SEQ ID No. 49).

In a preferred but non-limiting embodiment of the invention, the antibody comprises:

a) a heavy chain having CDR-H1, CDR-H2 and CDR-H3 of sequences SEQ ID No. 7, 2 and 3, respectively, and FR1, FR2 and FR3 derived from the human germ line IGHV1-46*01 (SEQ ID No. 46), and FR4 derived from the human germ line IGHJ4*01 (SEQ ID No. 48); and
b) a light chain having CDR-L1, CDR-L2 and CDR-L3 of sequences SEQ ID No. 9, 5 and 11, respectively, and FR1, FR2 and FR3 derived from the human germ line IGKV1-39*01 (SEQ ID No. 47), and FR4 derived from the human germ line IGKJ4*01 (SEQ ID No. 49).

In one embodiment, the antibody comprises a heavy chain variable domain (VH) of sequence SEQ ID No. 33 and a light chain variable domain (VL) of sequence SEQ ID No. 35. Hereinafter, said humanised antibody will be called hz208F2 (“Variant 1” or “Var. 1”).

In another embodiment, the antibody comprises a heavy chain variable domain (VH) of sequence SEQ ID No. 33, said sequence SEQ ID No. 33 comprising at least 1 back mutation chosen from among residues 20, 34, 35, 38, 48, 50, 59, 61, 62, 70, 72, 74, 76, 77, 79, 82 and 95.

In another embodiment, the antibody comprises a heavy chain variable domain (VH) of sequence SEQ ID No. 33, said sequence SEQ ID No. 33 comprising 2, 3, 4, 5, 6, 7, 8, 9, 10, 11, 12, 13, 14, 15, 16 or 17 back mutations chosen from among residues 20, 34, 35, 38, 48, 50, 59, 61, 62, 70, 72, 74, 76, 77, 79, 82 and 95.

For further clarity, Table 3 below illustrates the preferred back mutations.

TABLE 3

| | Residue no. | | | | | | | | |
|---|---|---|---|---|---|---|---|---|---|
| | 20 | 34 | 35 | 38 | 48 | 50 | 59 | 61 | 62 |
| Mouse | M | I | Y | K | L | W | K | N | E |
| Human | V | M | H | R | M | I | S | A | Q |
| | | Residue no. | | | | | | | |
| | | 70 | 72 | 74 | 76 | 77 | 79 | 82 | 95 |
| | Mouse | L | A | K | S | N | A | F | F |
| | Human | M | R | T | T | S | V | E | Y |

In one embodiment, the antibody comprises a light chain variable domain (VL) of sequence SEQ ID No. 35, said sequence SEQ ID No. 35 comprising at least 1 back mutation chosen from among residues 22, 53, 55, 65, 71, 72, 77 and 87.

In one embodiment, the antibody comprises a light chain variable domain (VL) of sequence SEQ ID No. 35, said sequence SEQ ID No. 35 comprising 2, 3, 4, 5, 6, 7 or 8 back mutations chosen from among residues 22, 53, 55, 65, 71, 72, 77 or 87.

In another embodiment, the antibody comprises:

a) a heavy chain variable domain (VH) of sequence SEQ ID No. 33, in which said sequence SEQ ID No. 33 comprises at least 1 back mutation chosen from among residues 20, 34, 35, 38, 48, 50, 59, 61, 62, 70, 72, 74, 76, 77, 79, 82 and 95; and
b) a light chain variable domain (VL) of sequence SEQ ID No. 35, said sequence of SEQ ID No. 35 comprising at least 1 back mutation chosen from among residues 22, 53, 55, 65, 71, 72, 77 and 87.

For further clarity, Table 4 below illustrates the preferred back mutations.

TABLE 4

| | Residue no. | | | | | | | |
|---|---|---|---|---|---|---|---|---|
| | 22 | 53 | 55 | 65 | 71 | 72 | 77 | 87 |
| Mouse | S | R | H | R | Y | S | N | F |
| Human | T | S | Q | S | F | T | S | Y |

In one such embodiment, the antibody comprises all the back mutations mentioned above and corresponds to an antibody comprising a heavy chain variable domain (VH) of sequence SEQ ID No. 34 and a light chain variable domain (VL) of sequence SEQ ID No. 36; Hereinafter, said humanised antibody will be called hz208F2 (“Variant 3” or “Var. 3”).

In another embodiment, all the humanised forms comprised between variant 1 and variant 3 are also encompassed by the present invention. In other words, the antibody is an antibody comprising a heavy chain variable domain (VH) of consensus sequence SEQ ID No. 41 and a light chain variable domain (VL) of consensus sequence SEQ ID No. 42. The humanised antibody in its entirety will hereinafter be called hz208F2 (“Variant 2” or “Var. 2”).

In a preferred but nonlimiting embodiment of the invention, the antibody is chosen from among:

a) an antibody comprising a heavy chain variable domain of sequence SEQ ID No. 33 or any sequence having at least 80%, preferably 85%, 90%, 95% and 98% identity with SEQ ID No. 33 and three light chain CDRs of sequences SEQ ID No. 9, 5 and 11; and
b) an antibody comprising a heavy chain variable domain of sequence SEQ ID No. 34 or any sequence having at least 80%, preferably 85%, 90%, 95% and 98% identity with SEQ ID No. 34 and three light chain CDRs of sequences SEQ ID No. 9, 5 and 11.

“Any sequence having at least 80%, preferably 85%, 90%, 95% and 98% identity with SEQ ID No. 33 or 34” means, respectively, sequences having three heavy chain CDRs SEQ ID No. 1, 2 and 3 and, moreover, having at least 80%, preferably 85%, 90%, 95% and 98%, identity with complete sequence SEQ ID No. 33 or 34 beyond the sequences corresponding to the CDRs (i.e., SEQ ID No. 1, 2 and 3).

In one embodiment of the invention, the antibody is chosen from among:

a) an antibody comprising a heavy chain variable domain of sequence SEQ ID No. 33 or any sequence having at least 80% identity with SEQ ID No. 33 and three light chain CDRs of sequences SEQ ID No. 9, 5 and 11; and
b) an antibody comprising a heavy chain variable domain of sequence SEQ ID No. 34 or any sequence having at least 80% identity with SEQ ID No. 34 and three light chain CDRs of sequences SEQ ID No. 9, 5 and 11.

If it is not indicated in the paragraphs concerned, in the present description, by any sequence or by a sequence having at least 80% of a particular sequence, it should be understood that said sequence has at least 80% and preferably 85%, 90%, 95% and 98% identity with the reference sequence. Whether or not these sequences contain CDR sequences, it is understood that sequences having at least these CDRs must be identical to the CDRs of the reference sequence, identities at 80%, preferably 85%, 90%, 95% and 98% with the complete sequence having to be calculated for the remaining sequence located outside the sequences corresponding to these CDRs.

In a preferred embodiment, the antibody is chosen from among:

a) an antibody comprising a light chain variable domain of sequence SEQ ID No. 35 or any sequence having at least 80%, preferably 85%, 90%, 95% and 98% identity with SEQ ID No. 35 and three heavy chain CDRs of sequences SEQ ID No. 7, 2 and 3; and
b) an antibody comprising a light chain variable domain of sequence SEQ ID No. 36 or any sequence having at least 80%, preferably 85%, 90%, 95% and 98% identity with SEQ ID No. 36 and three heavy chain CDRs of sequences SEQ ID No. 7, 2 and 3.

“Any sequence having at least 80%, preferably 85%, 90%, 95% and 98% identity with SEQ ID No. 35 or 36” designates sequences having three light chain CDRs SEQ ID No. 4, 5 and 6 and, moreover, having at least 80%, preferably 85%, 90%, 95% and 98%, identity with complete sequence SEQ ID No. 35 or 36 beyond the sequences corresponding to the CDRs (i.e., SEQ ID No. 4, 5 and 6).

In one embodiment of the invention, the antibody is chosen from among:

a) an antibody comprising a light chain variable domain of sequence SEQ ID No. 35 or any sequence having at least 80% identity with SEQ ID No. 35 and three heavy chain CDRs of sequences SEQ ID No. 7, 2 and 3; and
b) an antibody comprising a heavy chain variable domain of sequence SEQ ID No. 36 or any sequence having at least 80% identity with SEQ ID No. 36 and three heavy chain CDRs of sequences SEQ ID No. 7, 2 and 3.

The humanised antibodies described here can also be characterised by the constant domain and, more particularly, said humanised antibodies can be selected especially from among IgG1, IgG2, IgG3, IgM, IgA, IgD or IgE. More preferably, in the context of the present invention, said humanised antibodies are IgG1 or IgG4.

According to one embodiment of the invention, the antibody is a humanised antibody comprising VH and VL variable domains such as described above in the IgG1 format. More preferentially, said humanised antibody comprises a constant domain for the VH of sequence SEQ ID No. 43 and a kappa domain for the VL of sequence SEQ ID No. 45.

According to one embodiment of the invention, the antibody is a humanised antibody comprising VH and VL variable domains such as described above in the IgG4 format. More preferentially, said humanised antibody comprises a constant domain for the VH of sequence SEQ ID No. 44 and a kappa domain for the VL of sequence SEQ ID No. 45.

According to another embodiment of the invention, the antibody is chosen from among:

a) an antibody comprising or consisting of a heavy chain of sequence SEQ ID No. 37 or any sequence having at least 80% identity with SEQ ID No. 37 and a light chain of sequence SEQ ID No. 39 or any sequence having at least 80% identity with SEQ ID No. 39; and
b) an antibody comprising or consisting of a heavy chain of sequence SEQ ID No. 38 or any sequence having at least 80% identity with SEQ ID No. 38 and a light chain of sequence SEQ ID No. 40, or any sequence having at least 80% identity with SEQ ID No. 40.

For further clarity, Table 5a below illustrates non-limiting examples of VH and VL sequences for variant 1 (Var. 1) and variant 3 (Var. 3) of humanised antibody hz208F2. It also comprises the consensus sequence for variant 2 (Var. 2).

TABLE 5a

| | Heavy chain | Light chain | SEQ ID No. |
|---|---|---|---|
| hz208F2 | Variable domain (VH) | | 33 |
| (var. 1) | | Variable domain (VL) | 35 |
| | Complete length | | 37 |
| | | Complete length | 39 |
| hz208F2 | Variable domain (VH) | | 34 |
| (Var. 3) | | Variable domain (VL) | 36 |
| | Complete length | | 38 |
| | | Complete length | 40 |
| hz208F2 | Variable domain (VH) | | 41 |
| (Var. 2) | | Variable domain (VL) | 42 |

In another preferred embodiment, the antibody is chosen from among:

a) an antibody comprising a variable domain of heavy chain sequence chosen from among sequence SEQ ID No. 56, 62, 64, 66, 68, 70, 72, 74, 76, 78 and 54 or any sequence having at least 80%, preferably 85%, 90%, 95% and 98% identity with SEQ ID No. 56, 62, 64, 66, 68, 70, 72, 74, 76, 78 and 54 and three light chain CDRs of sequences SEQ ID No. 9, 5 and 11;
b) an antibody comprising a variable domain of light chain sequence chosen from among sequence SEQ ID No. 57 or 60 or any sequence having at least 80%, preferably 85%, 90%, 95% and 98% identity with SEQ ID No. 57 or 60 and three heavy chain CDRs of sequences SEQ ID No. 7, 2 and 3; and
c) an antibody comprising a variable domain of heavy chain sequence chosen from among SEQ ID No. 56, 62, 64, 66, 68, 70, 72, 74, 76, 78 and 54 or any sequence of at least 80%, preferably at least 85%, 90%, 95% and 98% identity with SEQ ID No. 56, 62, 64, 66, 68, 70, 72, 74, 76, 78 and 54; and a light chain variable domain chosen from among SEQ ID No. 57 or 60 or any sequence having at least 80%, preferably at least 85%, 90%, 95% and 98% identity with SEQ ID No. 57 or 60.

According to another embodiment of the invention, the antibody is chosen from among:

a) an antibody comprising a heavy chain of sequence SEQ ID No. 56, 62, 64, 66, 68, 70, 72, 74, 76, 78 and 54 or any sequence having at least 80% identity with SEQ ID No. 56, 62, 64, 66, 68, 70, 72, 74, 76, 78 or 54, and a light chain of SEQ ID No. 57 or any sequence having at least 80% identity with SEQ ID No. 57; and
b) an antibody comprising a heavy chain of sequence SEQ ID No. 56, 64, 68 and 78 or any sequence having at least 80% identity with SEQ ID No. 56, 64, 68 or 78 and a light chain of sequence SEQ ID No. 60, or any sequence having at least 80% identity with SEQ ID No. 60.

According to another embodiment of the invention, the antibody is chosen from among:

a) an antibody comprising or consisting of a heavy chain of sequence SEQ ID No. 58 or any sequence having at least 80% identity with SEQ ID No. 58 and a light chain of sequence SEQ ID No. 59 or any sequence having at least 80% identity with SEQ ID No. 59;
b) an antibody comprising or consisting of a heavy chain of sequence SEQ ID No. 58 or any sequence having at least 80% identity with SEQ ID No. 58 and a light chain of sequence SEQ ID No. 61, or any sequence having at least 80% identity with SEQ ID No. 61;
c) an antibody comprising or consisting of a heavy chain of sequence SEQ ID No. 63 or any sequence having at least 80% identity with SEQ ID No. 63 and a light chain of sequence SEQ ID No. 59, or any sequence having at least 80% identity with SEQ ID No. 59;
d) an antibody comprising or consisting of a heavy chain of sequence SEQ ID No. 65 or any sequence having at least 80% identity with SEQ ID No. 65 and a light chain of sequence SEQ ID No. 59 or any sequence having at least 80% identity with SEQ ID No. 59;
e) an antibody comprising or consisting of a heavy chain of sequence SEQ ID No. 65 or any sequence having at least 80% identity with SEQ ID No. 65 and a light chain of sequence SEQ ID No. 61, or any sequence having at least 80% identity with SEQ ID No. 61;
f) an antibody comprising or consisting of a heavy chain of sequence SEQ ID No. 67 or any sequence having at least 80% identity with SEQ ID No. 67 and a light chain of sequence SEQ ID No. 59, or any sequence having at least 80% identity with SEQ ID No. 59;
g) an antibody comprising or consisting of a heavy chain of sequence SEQ ID No. 69 or any sequence having at least 80% identity with SEQ ID No. 69 and a light chain of sequence SEQ ID No. 59 or any sequence having at least 80% identity with SEQ ID No. 59;
h) an antibody comprising or consisting of a heavy chain of sequence SEQ ID No. 69 or any sequence having at least 80% identity with SEQ ID No. 69 and a light chain of sequence SEQ ID No. 61, or any sequence having at least 80% identity with SEQ ID No. 61;
i) an antibody comprising or consisting of a heavy chain of sequence SEQ ID No. 71 or any sequence having at least 80% identity with SEQ ID No. 71 and a light chain of sequence SEQ ID No. 59, or any sequence having at least 80% identity with SEQ ID No. 59;
j) an antibody comprising or consisting of a heavy chain of sequence SEQ ID No. 73 or any sequence having at least 80% identity with SEQ ID No. 73 and a light chain of sequence SEQ ID No. 59 or any sequence having at least 80% identity with SEQ ID No. 59;
k) an antibody comprising or consisting of a heavy chain of sequence SEQ ID No. 75 or any sequence having at least 80% identity with SEQ ID No. 75 and a light chain of sequence SEQ ID No. 59 or any sequence having at least 80% identity with SEQ ID No. 59;
l) an antibody comprising or consisting of a heavy chain of sequence SEQ ID No. 77 or any sequence having at least 80% identity with SEQ ID No. 77 and a light chain of sequence SEQ ID No. 59, or any sequence having at least 80% identity with SEQ ID No. 59;
m) an antibody comprising or consisting of a heavy chain of sequence SEQ ID No. 53 or any sequence having at least 80% identity with SEQ ID No. 53 and a light chain of sequence SEQ ID No. 59 or any sequence having at least 80% identity with SEQ ID No. 59;
n) an antibody comprising or consisting of a heavy chain of sequence SEQ ID No. 53 or any sequence having at least 80% identity with SEQ ID No. 53 and a light chain of sequence SEQ ID No. 61, or any sequence having at least 80% identity with SEQ ID No. 61; and
o) an antibody comprising or consisting of a heavy chain of sequence SEQ ID No. 55 or any sequence having at least 80% identity with SEQ ID No. 55 and a light chain of sequence SEQ ID No. 59, or any sequence having at least 80% identity with SEQ ID No. 59.

In other words, the antibody can be an antibody comprising:

a) a heavy chain of sequence selected from among SEQ ID No. 58, 63, 65, 67, 69, 71, 73, 75, 77, 53 and 55 or any sequence having at least 80% identity with SEQ ID No. 58, 63, 65, 67, 69, 71, 73, 75, 77, 53 and 55; and
b) a light chain of sequence selected from among SEQ ID No. 59 and 61 or any sequence having at least 80% identity with SEQ ID No. 59 and 61.

In one embodiment of the invention, the antibody is chosen from among:

a) a heavy chain of sequence selected from among SEQ ID No. 58, 63, 65, 67, 69, 71, 73, 75, 77, 53 and 55 or any sequence having at least 80% identity with SEQ ID No. 58, 63, 65, 67, 69, 71, 73, 75, 77, 53 or 55; and
b) a light chain of sequence selected from among SEQ ID No. 59 and 61 or any sequence having at least 80% identity with SEQ ID No. 59 or 61.

For further clarity, Table 5b below illustrates non-limiting examples of VH and VL sequences (variable domain and complete length) for different variants of humanised antibody hz208F2.

TABLE 5b

| | Heavy chain | Light chain | SEQ ID NO. |
|---|---|---|---|
| hz208F2 | Variable domain (VH) | | 56 |
| H037/L018 | | Variable domain (VL) | 57 |
| | Complete length | | 58 |
| | | Complete length | 59 |
| Hz208F2 | Variable domain (VH) | | 56 |
| H037/L021 | | Variable domain (VL) | 60 |
| | Complete length | | 58 |
| | | Complete length | 61 |
| Hz208F2 | Variable domain (VH) | | 62 |
| H047/L018 | | Variable domain (VL) | 57 |
| | Complete length | | 63 |
| | | Complete length | 59 |
| Hz208F2 | Variable domain (VH) | | 64 |
| H049/L018 | | Variable domain (VL) | 57 |
| | Complete length | | 65 |
| | | Complete length | 59 |
| Hz208F2 | Variable domain (VH) | | 64 |
| H049/L021 | | Variable domain (VL) | 60 |
| | Complete length | | 65 |
| | | Complete length | 61 |
| Hz208F2 | Variable domain (VH) | | 66 |
| H051/L018 | | Variable domain (VL) | 57 |
| | Complete length | | 67 |
| | | Complete length | 59 |
| Hz208F2 | Variable domain (VH) | | 68 |
| H052/L018 | | Variable domain (VL) | 57 |
| | Complete length | | 69 |
| | | Complete length | 59 |
| Hz208F2 | Variable domain (VH) | | 68 |
| H052/L021 | | Variable domain (VL) | 60 |
| | Complete length | | 69 |
| | | Complete length | 61 |
| Hz208F2 | Variable domain (VH) | | 70 |
| H057/L018 | | Variable domain (VL) | 57 |
| | Complete length | | 71 |
| | | Complete length | 59 |
| Hz208F2 | Variable domain (VH) | | 72 |
| H068/L018 | | Variable domain (VL) | 57 |
| | Complete length | | 73 |
| | | Complete length | 59 |
| Hz208F2 | Variable domain (VH) | | 74 |
| H070/L018 | | Variable domain (VL) | 57 |
| | Complete length | | 75 |
| | | Complete length | 59 |
| Hz208F2 | Variable domain (VH) | | 76 |
| H071/L018 | | Variable domain (VL) | 57 |
| | Complete length | | 77 |
| | | Complete length | 59 |
| Hz208F2 | Variable domain (VH) | | 78 |
| H076/L018 | | Variable domain (VL) | 57 |
| | Complete length | | 53 |
| | | Complete length | 59 |
| Hz208F2 | Variable domain (VH) | | 78 |
| H076/L021 | | Variable domain (VL) | 60 |
| | Complete length | | 53 |
| | | Complete length | 61 |
| Hz208F2 | Variable domain (VH) | | 54 |
| H077/L018 | | Variable domain (VL) | 57 |
| | Complete length | | 55 |
| | | Complete length | 59 |

According to another aspect of the present invention, the antibody is an antibody produced by hybridoma 1-4757, 1-4773, 1-4775, 1-4736 or 1-4774 filed with the CNCM of the Institut Pasteur France on 30 May 2013, 26 Jun. 2013, 26 Jun. 2013, 24 Apr. 2013 and 26 Jun. 2013, respectively.

The antibody is more particularly a 208F2 antibody (for example, chimeric or humanised, especially chimeric) and more particularly a c208F2 antibody which is a monoclonal antibody (IgG1). This antibody is especially described in WO2015162291 and is characterised in that it comprises three light chain CDRs of SEQ ID No. 9, 5 and 11, respectively, and three heavy chain CDRs of SEQ ID No. 7, 2 and 3, respectively. The antibody is also characterised in that it comprises a light chain variable domain of sequence SEQ ID No. 18 and a heavy chain variable domain of sequence SEQ ID No. 13. The antibody is finally characterised in that it comprises a light chain of sequence SEQ ID No. 28 and a heavy chain variable domain of sequence SEQ ID No. 23.

Antibody c208F2 and ADC-A can be obtained by any methods known to the skilled person for obtaining antibodies and ADCs. They are especially obtained by the methods described in WO2015162291 (ADC-A is preferably the ADC called c208F2-G-13 in WO2015162291).

Treatment

According to the invention, “resistance” means that the patient no longer has a response to treatment, i.e., the cancer has started progressing again or is stable.

According to the invention, “treatment” especially means an increase in overall survival and/or an increase in the duration of progression-free survival and/or an increase in -free survival without worsening and/or a reduction in recurrence and/or a reduction in the size of the tumour.

The evaluation of these tumoral response criteria (resistance and treatment) is well known to the skilled person and can especially be measured by the RECIST criteria (Eisenhauer et al. Eur J Cancer, 45 (2009): 228-247 or its updates).

The proliferative disease can more particularly be a cancer, and especially a cancer resistant to docetaxel.

The cancer according to the invention can especially be selected from breast cancer, colon cancer, melanoma, lung cancer, including non-small cell lung cancer, stomach cancer, upper aerodigestive tract cancer, oesophageal cancer, colorectal cancer, stomach cancer, glioma, oesophageal cancer, ovarian cancer, prostrate cancer, renal cancer, thyroid cancer, uterine cancer, oral squamous cell carcinoma and mesothelioma.

In a particular embodiment, the cancer treated according to the invention will be a cancer usually treated with docetaxel, especially stomach, prostate, lung (including non-small cell), breast and upper aerodigestive tract cancer.

In a particular embodiment, the cancer treated according to the invention will be a cancer comprising tumour cells expressing or over-expressing all or part of the IGF-1R protein. Cancers expressing IGF-1R can be cancers initially expressing IGF-1R or cancers that did not initially express IGF-1R but which started to express it after becoming resistant to a first (or later) treatment, such as resistant to docetaxel.

In one embodiment, ADC-A may be administered after onset of resistance to docetaxel. ADC-A may thus be administered alone, subsequently to docetaxel or concomitantly with docetaxel and/or another chemotherapy. The initiation of treatment with ADC-A may especially start immediately after observation of the occurrence of resistance or in 1, 2, 3, 4, 5, 6, 7, or even 8 weeks or more after observation of the occurrence of resistance, especially before 16 weeks following the observation of the occurrence of resistance.

In another embodiment, ADC-A may be administered before onset of resistance to docetaxel, concomitantly with docetaxel. Treatment with ADC-A may then be initiated at the same time or after docetaxel treatment but before the onset of resistance to docetaxel.

“Concomitantly” means that the products are administered so as to be present in the patient's body at the same time. The products can be administered at the same time or sequentially.

“Subsequently” means that the initiation of treatment with the first product follows the treatment with the second product, for example that the treatment with ADC-A follows the treatment with docetaxel. The two products are therefore not simultaneously present in the patient's body.

The dosage regimen for docetaxel will depend on severity, patient condition and type of cancer to be treated. It may also vary depending on the prior or concomitant treatments received by the patient. Generally, docetaxel will be administered at a dose comprised between 50 mg/m² of body surface area (BSA) and 125 mg/m², especially between 75 mg/m² and 100 mg/m² every 1 to 4 weeks, especially every three weeks.

The number of treatment cycles (i.e., the number of occurrences of administration of the treatment and the time between two administrations) will depend on the patient's response and the onset of resistance to docetaxel. It may notably be comprised between 3 and 12 cycles or more.

The dosage regimen for ADC-A will depend on severity, patient condition and type of cancer to be treated. It may also vary depending on the prior or concomitant treatments received by the patient. Generally, ADC-A will be administered at a dose comprised between 0.5 and 3 mg/kg of the patient to be treated, especially between 0.9 and 2.5 mg/kg of the patient to be treated, every 1 to 4 weeks, especially every three weeks.

The number of treatment cycles with ADC-A will especially depend on the patient's response. It may especially be comprised between 1 and 18 cycles or more until resumption of progression of the tumour or remission thereof.

The treatment according to the invention may be combined with other cancer treatments prior to or concomitantly with docetaxel and/or ADC-A. In particular, the cancer treatment is treatment with a drug molecule chosen from the group consisting of doxorubicin, cyclophosphamide, capecitabine, cisplatin, paclitaxel, carboplatin, lapatinib, pertuzumab, bevacizumab, trastuzumab, 5-fluorouracil, anthracycline, vinorelbine, binimetinib, encorafenib and neratinib. The treatment according to the invention may also be combined with other types of medicaments such as corticosteroids, especially dexamethasone or prednisone.

The present invention also relates to a pharmaceutical composition comprising ADC-A and at least one pharmaceutically-acceptable excipient for its use according to the invention. In particular, ADC-A will be formulated so as to be able to be injected intravenously.

### EXAMPLE

Material and Method

Cell: MCF-7 cells are obtained from ATTC (Manassas). The cells are kept in an incubator at 5% CO₂, 90% humidity and 37° C. in a standard cell culture medium, as recommended by the supplier.

Antibody generation: Antibody 208F2 is produced as described in WO2015162291. Briefly, the anti-IGF-1R antibody, humanised c208F2 (hz208F2(4)) is generated by using hybridoma technology. Balb/c mice are immunised with recombinant human IGF-1R protein (rhIGF-1R) (R&D Systems) in the presence of Freund's adjuvant. The mice are fused with the myeloma SP2/0 fusion partner. After cloning by limiting dilution, the binding and internalisation of the antibody to MCF-7 cells is confirmed and the isotype determined. Binding specificity is verified by ELISA on rhIGF-1R, recombinant human insulin receptor (rhIR) and mouse IGF-1R (mIGF-1R) (R&D Systems). The antibody m208F2 is then humanised by grafting complementarity-determining regions (CDR) and expressed in Chinese hamster ovarian cells for a complete pharmacological characterisation.

Production and characterisation of ADC-A: ADC-A is produced as described in application WO2015162291 (ADC-A is the ADC called c208F2-G-13 in WO2015162291). A slight reduction of mAb 208F2 (hz208F2-4) and linker-payload coupling is carried out as described previously (Sun et al. Bioconjug. Chem 2005; 16:1282-90, Wagner-Rousset et al. mAbs 2014; 6:173-84). Briefly, to target a DAR of 4, hz208F2-4 was reduced with 2.5 molar equivalents of tris (2-carboxyethyl) phosphine (TCEP). The reduced antibody was then treated with 7 molar equivalents of linker-payload (G-13). After conjugation, ADC-A is concentrated to 5 mg/mL (in buffer in 25 mM of histidine pH 6.5, 150 mM NaCl and 6% sucrose). Tween 80 was then added to obtain a final concentration of 0.005% (v/v). The product is finally filtered through a Stericup filter (GP Express PLUS Membrane, 0.22 μm, Polyethersulfone, Millipore) and stored at 4° C.

Determination of in-vivo activity: In-vivo activity is tested in a breast cancer model: 7-week old nude female mice (Charles River Laboratories, n=15) were subcutaneously grafted with 5×10⁶ MCF-7 cells 1 day after subcutaneous implantation of 0.72 mg of granules releasing 17β-estradiol (Innovative Research of America) for 60 days.

The mice are randomised, and treatment is initiated when the tumours reach a size of approximately 150 mm³. The tumour volume (length×width×height×0.52) is measured at least twice a week and the therapeutic response is defined using solid tumour response criteria (RECIST).

Mice bearing MCF-7 tumours are injected intravenously with docetaxel, 9 mg/kg twice a week for a total of 5 injections. When the tumours become resistant to docetaxel, the mice are divided into 2 groups of 5 animals: group 1 receives 9 mg/kg of docetaxel every 2 weeks and group 2 receives a single injection of ADC-A of 3 mg/kg.

The percentage of the regression values is calculated using the following formula:

$$Regression \% = 100 \times Δ T / T initial$$

where T=the tumour volume in the treated group, ΔT=the tumour volume in the treated group on the day of the study minus the tumour volume in the treated group on the initial day of administration, and Tinitial=the tumour volume in the treated group on the initial day of the administration The disease is considered to be progressing when the tumour size increases by >20%. Partial regression (PR) is defined as a reduction in tumour size >30%. The absence of tumour growth, or a slight decrease (<30%), or a slight increase (<20%) of tumour size is defined as stable disease (SD), and an absence of palpable tumour mass is defined as complete regression (CR).

### Results

In order to evaluate the therapeutic potential of ADC-A in breast cancer, the effect of ADC-A administration was studied in a mouse model grafted with MCF-7 cells after onset of docetaxel resistance. After 70 days, the tumours became resistant to docetaxel and relapsed (FIG. 1). The administration of a single dose of ADC-A of 3 mg/kg in mice with docetaxel-resistant tumours induces strong and significant inhibition of tumour growth (p<0.05) (FIG. 1). CR was observed in 1 mouse and PR in 3 mice out of 5. ADC-A and docetaxel exhibit a synergistic effect. We can conclude that ADC-A is a good treatment alone or in combination with docetaxel for treating docetaxel-resistant tumours. This is all the more surprising given that the payload used has a similar mechanism of action (action on tubulin) to taxanes and therefore to docetaxel.

1-15. (canceled)

16. A method for treating a proliferative disease comprising the administration to a patient in need thereof of an effective quantity of docetaxel and an ADC of formula (I) below: 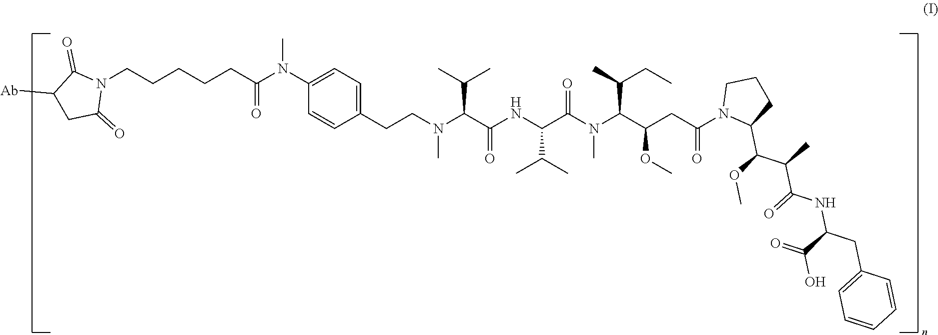
wherein Ab is an anti-IGF1R antibody comprising light chain CDRs of sequences SEQ ID Nos. 9, 5 and 11, respectively, and heavy chain CDRs of sequence SEQ ID Nos. 7, 2 and 3, respectively, and n is comprised between 1 and 12,
wherein the ADC is administered concomitantly with or subsequently to docetaxel.

17. The method according to claim 16, wherein the proliferative disease is cancer.

18. The method according to claim 16, wherein the Ab is an antibody comprising a light chain variable domain of sequence SEQ ID No. 18 and a heavy chain variable domain of sequence SEQ ID No. 13.

19. The method according to claim 16, wherein the patient has a resistance to docetaxel.

20. The method according to claim 16, wherein treating the proliferative disease with the ADC has a duration comprised between 1 month and 5 years.

21. The method according to claim 16, wherein the ADC is administered immediately after observation of an occurrence of resistance to docetaxel or in 1, 2, 3, 4, 5, 6, 7, or even 8 weeks or more after observation of the occurrence of resistance.

22. The method according to claim 21, wherein the ADC is administered before 16 weeks following the observation of the occurrence of resistance.

23. The method according to claim 16, wherein the ADC is administered by cycles at a dose comprised between 1 and 2 mg/kg of the patient every 2 to 4 weeks.

24. The method according to claim 23, wherein the ADC is administered by cycles with a number of the cycles being comprised between 1 and 18 cycles.

25. The method according to claim 16, wherein treating the proliferative disease is measured by an increase in overall survival, an increase in the duration of progression-free survival, an increase in survival without worsening, a reduction in recurrence, a reduction in the size of the tumour or a combination thereof.

26. The method according to claim 16, wherein the patient is treated by one or more other chemotherapies, prior to or concomitantly with docetaxel and/or with the ADC.

27. The method according to claim 26, wherein the one or more other chemotherapies comprise the administration of one or more drug molecules chosen from the group consisting of doxorubicin, cyclophosphamide, capecitabine, cisplatin, paclitaxel, carboplatin, lapatinib, pertuzumab, bevacizumab, trastuzumab, 5-fluorouracil, anthracycline, binimetinib, encorafenib and neratinib.

28. A method for treating a proliferative disease comprising the administration to a patient in need thereof of an effective quantity of docetaxel and a pharmaceutical composition comprising at least one pharmaceutically-acceptable excipient and an ADC of formula (I) below: 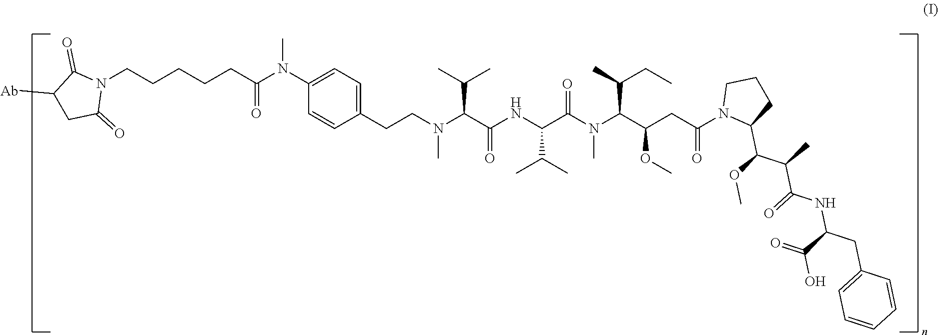
wherein Ab is an anti-IGF1R antibody comprising light chain CDRs of sequences SEQ ID Nos. 9, 5 and 11, respectively, and heavy chain CDRs of sequence SEQ ID Nos. 7, 2 and 3, respectively, and n is comprised between 1 and 12,
wherein the pharmaceutical composition comprising the ADC is administered concomitantly with or subsequently to docetaxel.

29. The method according to claim 28, wherein the Ab is an antibody comprising a light chain variable domain of sequence SEQ ID No. 18 and a heavy chain variable domain of sequence SEQ ID No. 13.

30. The method according to claim 28, wherein the proliferative disease is cancer.

31. The method according to claim 28, wherein the proliferative disease is a cancer resistant to docetaxel.

32. The method according to claim 28, wherein the pharmaceutical composition comprising the ADC is administered immediately after observation of an occurrence of resistance to docetaxel or in 1, 2, 3, 4, 5, 6, 7, or even 8 weeks or more after observation of the occurrence of resistance.

33. The method according to claim 32, wherein the pharmaceutical composition comprising the ADC is administered before 16 weeks following the observation of the occurrence of resistance.

34. The method according to claim 28, wherein the pharmaceutical composition comprising the ADC is administered by cycles at a dose of the ADC comprised between 1 and 2 mg/kg of the patient every 2 to 4 weeks.

35. The method according to claim 34, wherein the pharmaceutical composition comprising the ADC is administered by cycles with a number of the cycles being comprised between 1 and 18 cycles.

36. The method according to claim 28, wherein treating the proliferative disease with the pharmaceutical composition comprising the ADC has a duration comprised between 1 month and 5 years.

37. The method according to claim 28, wherein the patient is treated by one or more other chemotherapies, prior to or concomitantly with docetaxel and/or with the ADC.

38. The method according to claim 37, wherein the one or more other chemotherapies comprise the administration of one or more drug molecules chosen from the group consisting of doxorubicin, cyclophosphamide, capecitabine, cisplatin, paclitaxel, carboplatin, lapatinib, pertuzumab, bevacizumab, trastuzumab, 5-fluorouracil, anthracycline, binimetinib, encorafenib and neratinib.
